(19) United States
(12) Patent Application Publication
(10) Pub. No.: US 2013/0131089 A1
(43) Pub. Date: May 23, 2013

(54) DOSAGE FORMS OF ELINOGREL AND METHODS OF INJECTABLE ADMINISTRATION THEREOF

(71) Applicants: Hui Li, Sammamish, WA (US); Juan Wang, Foster City, CA (US); Joe Lambing, Burlingame, CA (US); Harry Tiemessen, Weil am Rhein (DE)

(72) Inventors: Hui Li, Sammamish, WA (US); Juan Wang, Foster City, CA (US); Joe Lambing, Burlingame, CA (US); Harry Tiemessen, Weil am Rhein (DE)

(73) Assignee: Portola Phamaceuticals Inc., South San Francisco, CA (US)

(21) Appl. No.: 13/695,428

(22) PCT Filed: May 2, 2011
(86) PCT No.: PCT/US11/34861

§ 371 Date: Jan. 14, 2013

Related U.S. Application Data
(63) Continuation-in-part of application No. 61/329,725, filed on Apr. 30, 2010.

(51) Int. Cl. A61K 9/00

(52) U.S. Cl. CPC A61K 9/0019
USPC 514/266.24

(57) ABSTRACT

The present invention is concerned with a liquid delivery form of Elinogrel for the treatment of thrombosis which is notable for its improved dosage properties and stability. The dosage form is a liquid or a lyophilized form which is reconstituted for an injectable formulation comprising: a) at least about 3 mg/ml or up to about 15 mg/ml Elinogrel or a pharmaceutically acceptable salt thereof (post reconstitution or in liquid form), and b) at least one pharmaceutically acceptable excipient. Further aspects of the present invention concern the preparation and use of such a formulation.

### CROSS-REFERENCES TO RELATED APPLICATIONS

This application claims priority benefit of U.S. Provisional Application Ser. No. 61/329,725 filed on Apr. 30, 2010, the contents of which are incorporated herein by reference in its entirety for all purposes.

### TECHNICAL FIELD OF INVENTION

The present invention relates to compositions for the delivery of pharmacologically active agents by injection and to methods of treating and/or preventing disease in mammals, particularly humans, by administering a pharmacologically active agent in accordance with the invention.

### BACKGROUND OF THE INVENTION

Delivery of pharmacologically active agents by injection is generally the delivery route of choice when oral delivery is not practical or an immediate therapeutic activity is required. However, biological, chemical and physical barriers such as poor solubility make delivery of some pharmacologically active agents by injection to mammals problematic. U.S. patent application Ser. No. 11/556,490 filed Nov. 3, 2006 discloses compounds having the formula (I):

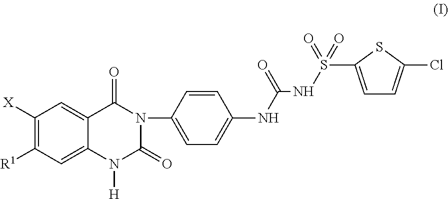

wherein: R¹ is selected from the group consisting of H, halogen, —OH, —C₁₋₁₀-alkyl and C₁₋₆-alkylamino; and X is selected from the group consisting of: F and I; for example, are being developed for the treatment of thrombotic complications.

Thrombotic complications are a major cause of death in the industrialized world. Examples of these complications include acute coronary syndrome (ACS), acute myocardial infarction, unstable angina, chronic stable angina, transient ischemic attacks, strokes, peripheral vascular disease, preeclampsia/eclampsia, deep venous thrombosis, embolism, disseminated intravascular coagulation and thrombotic cytopenic purpura. Thrombotic and restenotic complications also occur following invasive procedures, e.g., angioplasty, carotid endarterectomy, post CABG (coronary artery bypass graft) surgery, vascular graft surgery, stent placements and insertion of endovascular devices and prostheses, and hypercoagulable states related to genetic predisposition or cancers. It is generally thought that platelet aggregates play a critical role in these events. Blood platelets, which normally circulate freely in the vasculature, become activated and aggregate to form a thrombus from disturbed blood flow caused by ruptured atherosclerotic lesions or by invasive treatments such as angioplasty, resulting in vascular occlusion. Platelet activation can be initiated by a variety of agents, e.g., exposed subendothelial matrix molecules such as collagen, or by thrombin which is formed in the coagulation cascade.

### [4-(6-Fluoro-7-methylamino-2,4-dioxo-1,4-dihydro-2H-quinazolin-3-yl)-phenyl]-5-chloro-thiophen-2-yl-sulfonylurea (Elinogrel)

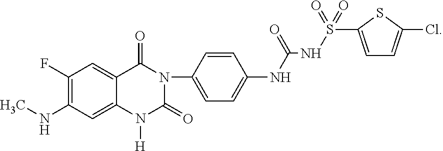 [SMILES: CNc1cc2[nH]c(=O)n(-c3ccc(NC(=O)NS(=O)(=O)c4ccc(Cl)s4)cc3)c(=O)c2cc1F]

As its potassium salt, Elinogrel has a molecular weight of 562.04 (free acid 523.95). Its pKa is about 3.3 with a logP of about 2.5 and logD (pH 7.4) of about −1.6. Formulation of Elinogrel has proven difficult due, at least in part, to the poor aqueous solubility of the free acid form which is <0.1 mg/ml (i.e. practically insoluble) at pH 1.0-7.4. Techniques have been disclosed for preparing sustained (or controlled) release pharmaceutical formulations of Elinogrel (see e.g. U.S. patent application Ser. No. 12/618,511 filed Nov. 13, 2009.

The thermodynamic solubility of a compound can be lower than the concentration of the compound after reconstitution from a lyophilized formulation. Reconstitution of the previous lyophilized intravenous (IV) formulations and storage could potentially cause precipitation especially if seed crystals exist or form in solution. Therefore, there exists a continuing need for further improvement in injectable preparations which accommodate higher clinical doses and provide still greater unit dose of Elinogrel, and other weakly acidic drugs, or their pharmaceutically acceptable salts. Accordingly, there is a significant need for highly concentrated liquid formulations of these compounds, e.g. up to about 15 mg/ml which avoid problems of instability due to the formation of particulates or other components which make further processing difficult. In addition, there is a significant need for highly concentrated liquid formulations of these compounds, e.g. up to about 15 mg/ml which allow lower volumes to be injected into a patient. The present invention satisfies these and other needs. It has now been found, in accordance with the present invention, that an injectable pharmaceutical formulations which accommodates higher clinical doses and provide still greater unit doses of Elinogrel can be obtained.

### BRIEF SUMMARY OF THE INVENTION

Accordingly, the present invention is directed to pharmaceutical compositions administrable by injection which, unexpectedly, greatly enhances the unit dose of a weakly acidic drug compound or a pharmaceutically acceptable salt thereof, with poor aqueous solubility, such as Elinogrel. Specifically in one aspect, the invention provides a stable, highly concentrated liquid pharmaceutical composition comprising at least about 3 mg/mL Elinogrel, or a pharmaceutically acceptable salt thereof. In another aspect, the invention provides a lyophilized pharmaceutical composition which comprises at least about 3 mg/ml Elinogrel, or a pharmaceutically acceptable salt thereof after reconstitution in solution.

In a further aspect, the invention is directed to a dosage form comprising: a liquid or lyophilized pharmaceutical composition comprising: a) at least about 3 mg/ml Elinogrel or a pharmaceutically acceptable salt thereof, and b) at least one pharmaceutically acceptable excipient.

In a still further aspect, the invention is directed to a method of treating or preventing a thrombotic conditions in a mammal in need thereof, which method comprises administering to said mammal an effective amount of a liquid pharmaceutical composition comprising: a) at least about 3 mg/ml Elinogrel or a pharmaceutically acceptable salt thereof, and b) at least one pharmaceutically acceptable excipient.

In another aspect the present invention provides a method to aid in and keeping Elinogrel, or its pharmaceutically acceptable salts thereof, miscible and inhibiting crystallization of Elinogrel, or a pharmaceutically acceptable salt thereof Elinogrel, in solution comprising the step of: providing a suitable pharmaceutically acceptable solubilizer or crystallization inhibitor. In one aspect the solubilizer or crystallization inhibitor is sodium sulfobutyl ether β-cyclodextrin (SBE-beta-CD, CAPTISOL®), or pharmaceutically acceptable salts thereof or combinations thereof

In a further aspect the present invention provides a method of producing a lyophilized pharmaceutical composition comprising at least about 3 mg/mL Elinogrel post reconstitution, or a pharmaceutically acceptable salt; by contacting a) Elinogrel or a pharmaceutically acceptable salt thereof, with b) at least one pharmaceutically acceptable excipient.

Another aspect of the present invention relates to a method for producing the liquid or lyophilized formulation.

Further features and advantages of the invention will become apparent from the following detailed description of the invention.

### BRIEF DESCRIPTION OF THE DRAWINGS

FIG. 1 shows the kinetic solubility of Forms B & C of Elinogrel potassium in a liquid formulation containing mannitol and glycine buffer.

FIG. 2 shows the thermodynamic solubility of Elinogrel as a free acid and as a potassium salt at different pH (adjusting pH with either NaOH or HCl) at 37° C.

### DETAILED DESCRIPTION OF THE INVENTION

### Definitions

As used herein, the following terms have the meanings ascribed to them unless specified otherwise.

The singular forms “a,” “an,” and, “the” include plural references unless the context clearly dictates otherwise. Thus, for example, reference to a compound refers to one or more compounds or at least one compound. As such, the terms “a” (or “an”), “one or more”, and “at least one” can be used interchangeably herein.

The phrase “about” as used herein is used to provide flexibility to a numerical range endpoint by providing that a given value may be “a little above” or “a little below” the endpoint accounting for variations one might see in measurements taken among different instruments, samples, and sample preparations.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. The terms “pharmaceutical formulation” as used herein refer to a pharmaceutical composition comprising the active ingredient and optionally further excipients to make it suitable to apply to a patient and is usually a finished drug product. The term “pharmaceutical formulation” and “pharmaceutical composition” may be interchangeably used herein depending on the context of these terms.

The terms “therapeutic agent,” “active agent,” “active compound,” or in some cases “compound,” “bioactive agent,” “pharmaceutically active agent,” and “pharmaceutical,” and “drug” are used interchangeably herein to refer to a substance having a pharmaceutical, pharmacological, psychosomatic, or therapeutic effect. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, co-crystals, and salts and solvates of co-crystals, etc. including crystal modifications such as polymorphs and amorphous forms etc. Suitable agents for use in the present invention include, without limitation, compounds which have the formula (I):

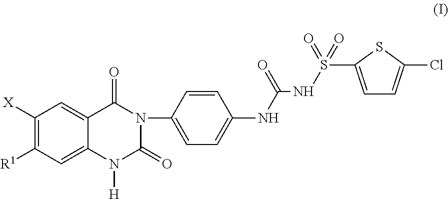

wherein:
R¹ is selected from the group consisting of H, halogen, —OH, —C₁₋₁₀-alkyl and C₁₋₆-alkylamino; and
X is selected from the group consisting of: F and I, or a pharmaceutically acceptable salt thereof; and combinations thereof. In a particularly preferred embodiment, the active agent is in a salt form such as that shown below, where the symbol M represents a suitable counterion, such as an alkali metal.

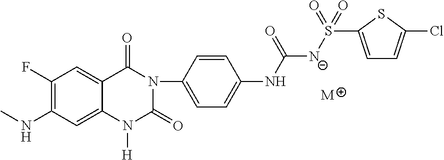

In a particularly preferred embodiment, the active agent is [4-(6-fluoro-7-methylamino-2,4-dioxo-1,4-dihydro-2H-quinazolin-3-yl)-phenyl]-5-chloro-thiophen-2-yl-sulfonylurea, (also referred to as 5-chloro-N-(4-(6-fluoro-7-(methylamino)-2,4-dioxo-1,2-dihydro-2H-quinazolin-3(4H)-yl)-phenylcarbamoyl)-thiophene-2-sulfonamide or Elinogrel), according to the structural formulae below:

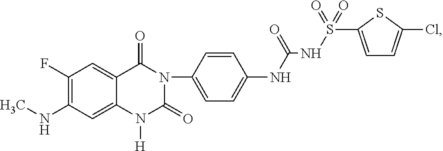 [SMILES: CNc1cc2[nH]c(=O)n(-c3ccc(NC(=O)NS(=O)(=O)c4ccc(Cl)s4)cc3)c(=O)c2cc1F]

in all suitable forms as disclosed for example in U.S. patent application Ser. No. 11/556,490 filed Nov. 3, 2006.

The present invention is applicable not only to [4-(6-fluoro-7-methylamino-2,4-dioxo-1,4-dihydro-2H-quinazolin-3-yl)-phenyl]-5-chloro-thiophen-2-yl-sulfonylurea, but also to other weakly acidic drugs with poor aqueous solubility.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

As used herein, the term “treating” refers to providing an appropriate dose of a therapeutic agent to a subject suffering from an ailment.

As used herein, the term “condition” refers to a disease state for which the compounds, compositions and methods of the present invention are being used against.

As used herein, the term “ADP-mediated disease or condition” and the like refers to a disease or condition characterized by less than or greater than normal, ADP activity. An ADP -mediated disease or condition is one in which modulation of ADP results in some effect on the underlying condition or disease (e.g., a ADP inhibitor or antagonist results in some improvement in patient well-being in at least some patients).

As used herein, “subject” refers to a mammal that may benefit from the administration of a drug composition or method of this invention. Examples of subjects include humans, and may also include other animals such as horses, pigs, cattle, dogs, cats, rabbits, rats, mice and aquatic mammals. In one specific aspect, a subject is a human.

As used herein, an “effective amount” or a “therapeutically effective amount” of a drug refers to a non-toxic, but sufficient amount of the drug, to achieve therapeutic results in treating a condition for which the drug is known to be effective, especially a thrombotic condition. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” or “excipient” may be used interchangeably, and refer to any inert and pharmaceutically acceptable material that has substantially no biological activity, and makes up a substantial part of the formulation.

A “stable” formulation is one in which the compounds therein essentially retain its physical and chemical stability and integrity upon storage. Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of precipitation during storage can be used as an indicator of stability. Thus, a “stable” formulation may be one wherein less than about 10% and preferably less than about 5% of the compound precipitates from the formulation.

A “reconstituted” formulation is one which has been prepared by dissolving a lyophilized formulation in a diluent such that the compound throughout. The reconstituted formulation is suitable for administration (e.g. parenteral administration) to a patient to be treated with the compound of interest and, in certain embodiments of the invention, may be one which is suitable for subcutaneous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present invention are hypertonic as a result of the addition of salt and/or buffer.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the terms “administration,” and “administering” refer to the manner in which an active agent is presented to a subject. While much of the disclosure is focused on injectable administration, administration can be accomplished by other various art-known routes that use liquid or lyophilized formulations such as transdermal, inhalation, implantation, ocular, otic, etc.

The term “injectable administration” represents any method of administration in which an active agent can be administered through injecting the liquid dosage form. Such liquid or lyophilized dosage forms are traditionally intended to immediately release and or deliver the active agent in the bloodstream. Examples of injection include intravenous (bolus and/or infusion), perivascular, subcutaneous, intramuscular, parenteral or intraperitoneal, etc.

As used herein, “injectable dosage form” refers to a formulation that is prepared for administration to a subject through an injectable route of administration. Examples of known liquid or lyophilized dosage forms, include without limitation, solutions, suspensions, solutions and solution pre-concentrates, emulsions and emulsion pre-concentrates, etc. The dosage forms of the present invention may be unit dosage forms wherein the dosage form is intended to deliver one therapeutic dose per administration.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### Description of the Embodiments

In one group of embodiments, the invention provides a liquid pharmaceutical composition comprising: a) at least about 3 mg/mL Elinogrel or a pharmaceutically acceptable salt thereof, and b) at least one pharmaceutically acceptable excipient. In another group of embodiments, the invention provides a lyophilized pharmaceutical composition comprising at least about 3 mg/ml Elinogrel, or a pharmaceutically acceptable salt thereof after reconstitution in solution.

In another group of embodiments, the invention provides a method of treating or preventing a thrombotic condition in a mammal in need thereof, which method comprises intravenous administering to said mammal an effective amount of a liquid pharmaceutical composition comprising at least about 3 mg/ml Elinogrel.

In another group of embodiments, the invention provides a method of producing a liquid pharmaceutical composition comprising at least about 3 mg/ml Elinogrel, or a pharmaceutically acceptable salt thereof; by contacting a) Elinogrel or a pharmaceutically acceptable salt thereof in solution, with b) at least one pharmaceutically acceptable excipient.

In another group of embodiments, the invention provides a method of producing a lyophilized pharmaceutical composition comprising at least about 3 mg/ml Elinogrel, or a pharmaceutically acceptable salt thereof after reconstitution; comprising a) forming a solution comprising at least about 3 mg/ml Elinogrel in an aqueous solvent, optionally a co-solvent and a bulking agent and (b) lyophilizing said solution to form a lyophilized pharmaceutical composition.

The composition provides a desired dose for the active agent under physiological conditions.

### Active Agents

In one set of embodiments, the active agents of the present invention are selected from the class of compounds in the dihydroquinazolinylphenyl thiophenyl sulfonylurea family and are useful in the treatment of conditions such as thrombosis. Illustrative examples of suitable dihydroquinazolinylphenyl thiophenyl sulfonylurea compounds for use in the present invention have the formula (I):

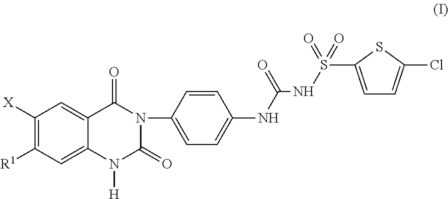

wherein:
R¹ is selected from the group consisting of H, halogen, —OH, —C₁₋₁₀-alkyl and C₁₋₆-alkylamino; and
X is selected from the group consisting of: F and I.

More preferably, the agent is [4-(6-fluoro-7-methylamino-2,4-dioxo-1,4-dihydro-2H-quinazolin-3-yl)-phenyl]-5-chloro-thiophen-2-yl-sulfonylurea, in all suitable forms. In one aspect, the invention provides a liquid or lyophilized composition, wherein the active agent is [4-(6-fluoro-7-methylamino-2,4-dioxo-1,4-dihydro-2H-quinazolin-3-yl)-phenyl]-5-chloro-thiophen-2-yl-sulfonylurea potassium or sodium salt. Methods for the preparation of compounds of formula (I) are described in US-2007-0123547-A1 and US-2009-0042916-A1.

It was found that compounds of formula (I) are weak acids with poor aqueous solubility at acidic pH. Thus, in one embodiment, the active agents of the present invention are a poorly soluble weak acid compound in its salt form that has aqueous solubility of less than 0.1 mg/ml at pH 1.0 -7.4 at a temperature of about 37° C. having an ionized form and an un-ionized form. The aqueous solubility increases at a higher pH (e.g. ≧1 mg/ml at pH 8 or above). In certain instances, the active agent is initially present at least partly in an ionized form. In certain other instances, the active agent is initially present in an un-ionized form. In one embodiment and as described in more detail below, the excipients of the compositions described herein helps to increase the solubility of the active as pH increases up to pH 10 in a hydrated media. In another embodiment, the excipient of the compositions described herein helps to maintain substantially all of the active agent in its dissolved ionized form in the formulation when it is in a hydrated media.

In another set of embodiments, the active agents of the present invention are any weakly acidic drug, or a pharmaceutically acceptable salt thereof with poor aqueous solubility.

As used herein, the term “active agent” includes all pharmaceutically acceptable forms of the active agent being described. For example, the active agent can be in a isomeric mixture, a solid complex bound to an ion exchange resin, or the like. In addition, the active agent can be in a solvated form. The term “active agent” is also intended to include all pharmaceutically acceptable salts, derivatives, and analogs of the active agent being described, as well as combinations thereof. For example, the pharmaceutically acceptable salts of the active agent include, without limitation, the sodium, potassium, calcium, magnesium, ammonium, tromethamine, L-lysine, L-arginine, N-ethylglucamine, N-methylglucamine and salt forms thereof, as well as combinations thereof and the like. Any form of the active agent is suitable for use in the compositions of the present invention, e.g., a pharmaceutically acceptable salt of the active agent, a free acid of the active agent, or a mixture thereof. In one embodiment, the invention provides a liquid or lyophilized composition, wherein the active agent is [4-(6-fluoro-7-methylamino-2,4-dioxo-1,4-dihydro-2H-quinazolin-3-yl)-phenyl]-5-chloro-thiophen-2-yl-sulfonylurea potassium salt having a crystalline solid form A or B or [4-(6-fluoro-7-methylamino-2,4-dioxo-1,4-dihydro-2H-quinazolin-3-yl)-phenyl]-5-chloro-thiophen-2-yl-sulfonylurea sodium salt as described in US-2007-0123547-A1. In another embodiment, the invention provides a liquid or lyophilized composition, wherein the active agent is [4-(6-fluoro-7-methylamino-2,4-dioxo-1,4-dihydro-2H-quinazolin-3-yl)-phenyl]-5-chloro-thiophen-2-yl-sulfonylurea sodium salt having a crystalline solid form A or B as described in US-2009-0042916-A1.

The active agent initially can be in any suitable form. In certain embodiments, the active agent can initially be in the form of an amorphous solid, a crystal, a granule, or a pellet.

In one group of embodiments, the invention provides a liquid or lyophilized pharmaceutical composition comprising: a) at least about 3 mg/mL Elinogrel or a pharmaceutically acceptable salt thereof, and b) at least one pharmaceutically acceptable excipient.

In another group of embodiments, the liquid pharmaceutical formulation comprises about 4 to about 25 mg/ml Elinogrel, or a pharmaceutically acceptable salt thereof. In another group of embodiments, the liquid or lyophilized pharmaceutical formulation comprises about 15 to 23 mg/ml Elinogrel, or a pharmaceutically acceptable salt thereof. In another group of embodiments, the liquid or lyophilized pharmaceutical formulation comprises about 17 mg/ml Elinogrel, or a pharmaceutically acceptable salt thereof. In another group of embodiments, the liquid or lyophilized pharmaceutical formulation comprises up to about 15 mg/ml Elinogrel free acid, or a pharmaceutically acceptable salt thereof. In another group of embodiments, the liquid or lyophilized pharmaceutical formulation comprises about 10 to about 15 mg/ml Elinogrel free acid, or a pharmaceutically acceptable salt thereof. In one embodiment, the formulation comprises about 8 to about 20 mg per ml Elinogrel, sodium salt.

In another group of embodiments, the invention provides a liquid or lyophilized injectable dosage form comprising: a) at least about 3 mg/ml Elinogrel or a pharmaceutically acceptable salt thereof, and b) at least one pharmaceutically acceptable excipient. In another group of embodiments, the invention provides a liquid or lyophilized injectable dosage form comprising Elinogrel or a pharmaceutically acceptable salt thereof in a unit dosage of from between about 10 and 150 mg. In another group of embodiments, the invention provides a liquid or lyophilized injectable dosage form comprising Elinogrel or a pharmaceutically acceptable salt thereof in a unit dosage of about 10, 20, 30, 40, 50, 60, 70, 80, 90, 100, 110 or 120 mg.

In another group of embodiments, the invention provides a liquid or lyophilized injectable dosage form produced from the free acid of Elinogrel. In another group of embodiments, the invention provides a liquid or lyophilized injectable dosage form wherein the Elinogrel is the potassium salt of Elinogrel. In another group of embodiments, the invention provides a liquid or lyophilized dosage form wherein the Elinogrel is the sodium salt of Elinogrel.

### Other Components and of the Formulations and Dosage Forms

### Excipients

One issue in using low solubility drugs is the crystallization of the drug in solution. The formulations and dosage forms described herein offer advantages over other traditional formulations and dosage forms for injectable administration for low solubility drugs,

Thus, in one group of embodiments, the liquid or lyophilized formulation comprises an excipient. In another group of embodiments, the excipient of the present invention is a solubilizer. As used herein, the term “solubilizer” refers to salts, ions, carbohydrates, surfactants, complexation agent, polymers and other compounds which, when present in solution, decrease the crystallization of Elinogrel. Examples of complexation agent include, but are not limited to sodium sulfobutyl ether f3-cyclodextrin and hydroxypropyl β-cyclodextrin, and pharmaceutically acceptable salts thereof and combinations thereof.

In one group of embodiments, the excipient is present in an amount of about 1% to about 10% by weight. In another group of embodiments, the excipient is present in an amount of about 4% by weight.

### Solvents and Co-Solvents

The formulations of the present invention can be for instance in form of an aqueous solutions. By “aqueous solutions” is meant a solution with the active ingredient and optional pharmaceutical excipients are dissolved using water as the principal solvent. The water may be buffered to stabilize the pH with a suitable buffer such as e.g. a phosphate buffer, acetate, citrate, lactate, glycine, tris, sodium bicarbonate, maleate buffer. The buffer is preferably present in suitable amount to adjust the pH to the desired value, e.g. in a concentration of 1 mM to 50 mM. The “aqueous solution” may further contain a water-miscible organic solvent or solvents. When an organic co-solvent is employed it is preferred that it is used in amounts of up to 75% by weight total solution, e.g. 0.01 to 75%. Suitable solvents are those water-miscible solvents commonly used in the art, for example propyleneglycol, polyethyleneglycol 300, polyethyleneglycol 400, glycerol, tween 20, tween 80 and ethanol.

The formulations suitable for administration are often formulated to have the approximately same osmotic pressure as body fluid such as e.g. blood. Accordingly, a formulation of the invention may comprise an isotonic agent which has the effect of rendering the osmotic pressure of the formulation the same as that of body fluid. The isotonic agent may be selected from any of those commonly used in the art, e.g. sucrose, mannitol, trehalose, glycine, sodium chloride, dextran and glucose. The isotonic agents may be used in quantities which impart to the formulation the same osmotic pressure as body fluid. The precise amount necessary to achieve the desired effect may depend on factors such as the concentration of active agent in the formulation, and is a matter of routine experimentation which the skilled person may determine without exercising any inventive thought and using only common general knowledge. Selection of the isotonic agent is preferably made having regard to the properties, e.g. stability of the active agent.

The pH of the formulation of the present invention is typically maintained in the range of about 4 to 10, or more preferably in the range of about 6 to 9.5. In one embodiment, the pH is at least about 9.0. In one embodiment, the pH is about 9.0 to about 9.1, 9.2, 9.3, 9.4, 9.5, 9.6, 9.7 or 9.8. In one embodiment, the pH is about 9.1 to about 9.2, 9.3, 9.4, 9.5, 9.6, 9.7 or 9.8. In one embodiment, the pH is about 9.2 to about 9.2, 9.3, 9.4, 9.5, 9.6, 9.7 or 9.8. In another group of embodiments, the pH of the liquid or lyophilized pharmaceutical formulation is about 9.0 to about 9.5. In one embodiment the pH of the liquid or lyophilized pharmaceutical formulation is about 9.1 to about 9.4. In one embodiment the pH of the liquid or lyophilized pharmaceutical formulation is about 9.3.

In one group of embodiments, the liquid (or in some instances the lyophilized) pharmaceutical formulation comprises a cosolvent. As used herein, a nonvolatile cosolvent refers to a substance having a vapor pressure lower than 0.50 mm Hg at 25° C. The purpose of the nonvolatile cosolvent is to facilitate the dissolution of a poorly water-soluble therapeutic compound in water in order to form a solution. Examples of a nonvolatile cosolvent include, without limitation, alkylene glycols such as, liquid PEG MW200-800, propylene glycol, polyhydric alcohols, e.g., mannitol, sorbitol and xylitol; polyoxyethylenes; linear polyols, e.g., ethylene glycol, 1,6-hexanediol, neopentyl glycol and methoxypolyethylene glycol; and mixtures thereof. In another group of embodiments, the cosolvent is selected from the group consisting of propylene glycol and ethanol.

### Other Components

Formulations according to the invention may contain other excipients commonly employed in injectable formulations in order to provide the required stability and therapeutic efficacy. Excipients may include EDTA as chelating agent or antioxidants such as, e.g., alpha-tocopherol, BHT, BHA and any other excipients commonly used in the preparation of injectable formulations. Antioxidants may be selected from any of those compounds known in the art. The amount of other suitable excipients employed can be determined using only routine experimentation.

As used herein, the term “bulking agent” refers to an ingredient that provides bulk to the pharmaceutical composition. Examples of bulking agents include, without limitation, mannitol, trehalose, lactose, sucrose, polyvinyl pyrrolidone, sucrose, glucose, glycine, cyclodextrins, dextran, solid PEGs and derivatives and mixtures thereof. A particularly useful bulking agent in accordance with the present invention is mannitol.

As will be appreciated by the skilled person, some components of the formulation can serve more than one function. Mannitol for instance may be used as isotonic agent, but may also act as bulking agent for lyophilization.

Surfactants can also be optionally used in the pharmaceutical composition. Examples of surfactants include fatty acid and alkyl sulfonates; benzethanium chloride, e.g., HYAMINE 1622 from Lonza, Inc. (Fairlawn, N.J.); polyoxyethylene sorbitan fatty acid esters, e.g., the TWEEN Series from Uniqema (Wilmington, Del.); and natural surfactants, such as sodium taurocholic acid, 1-palmitoyl-2-Sn-glycero-3-phosphocholine, lecithin and other phospholipids. Such surfactants, e.g., minimize aggregation of lyophilized particles during reconstitution of the product. These surfactants may comprise from about 0.001% to about 5% w/v.

The formulation further may comprise pH adjusting agents or buffering agents. In one embodiment, the pH adjusting agents or buffering agents of the compositions described herein are capable of raising the pH of the hydrated formulation to typically about 9.0-10.0, irrespective of the starting pH of media. In this way, the pH adjusting agents help increase the solubility of the active as pH increases up to pH 10 in a hydrated media to enhance the product release/dissolution profile from the hydrated formulation. Although pH adjusting agents or buffering agents may be used with the actives and excipients of the present invention, one skilled in the art will appreciate that acidic agents can also be used to adjust the pH of the pH adjusting agents or buffering agents as long as the pH adjusting agents or buffering agents as a whole raises the pH of the environment for these compounds in the hydrated formulation to greater than about the pKa of the active acid.

Suitable pH adjusting agents or buffering agents include, but are not limited to, organic and inorganic basic compounds of a wide range of aqueous solubilities and molecular weights and the like and mixtures thereof. Representative examples of inorganic basic salts include ammonium hydroxide, alkali metal salts, alkaline earth metal salts such as magnesium oxide, magnesium hydroxide, calcium hydroxide, sodium hydroxide, potassium hydroxide, aluminum hydroxide, potassium carbonate, sodium bicarbonate and the like and mixtures thereof. The solubility and the molecular size of the pH adjusting agent may affect its diffusion rate in the hydrated product and influence the dissolution profile of the active agent. In one group of embodiments, the pH adjusting agents or buffering agents are selected from the group consisting of glycine sodium and sodium hydroxide. Other commonly used buffer types for pH 7-10, which are not listed above include, but are not limited to glycine, sodium bicarbonate, tris, borax, diethanolamine, glutamic acid, triethanolamine and the like.

In one group of embodiments, the pH adjusting agents or buffering agent is present in a concentration of about 1 mM to about 50 mM. In another group of embodiments, the pH adjusting agents or buffering agent is present in a concentration of about 5 mM to about 10 mM. In one group of embodiments, the pH adjusting agent is glycine present in a concentration of at least about 0.75 mg/ml or 1mM or 2 mM. In another group of embodiments, the pH adjusting agent is glycine present in a concentration of about 1mM to 50mM; or 5 mM to 10 mM.

In one aspect, the invention provides a liquid or lyophilized composition wherein amount of pH adjusting agents or buffering agent is from about 1 to about 20 percent of the total composition. In one aspect, the invention provides a liquid or lyophilized composition wherein the combined percent of the pH adjusting agents or buffering agent is greater than or equal to the percent of the active. In one aspect, the invention provides a liquid or lyophilized composition wherein the amount of pH adjusting agents or buffering agent is from about 2 to about 15%. In one aspect, the invention provides a liquid or lyophilized composition wherein said composition comprises from about 3% to about 10% of the total composition.

### Preparation of Liquid and Lyophilized Compositions Comprising Active Agent

Water is weighed and added to a stainless steel vessel. Glycine and mannitol are added and mixed to dissolve. The Active Agent is added and mixed to dissolve. In one embodiment the active ingredient is Elinogrel sodium. pH is adjusted to final pH by addition of a solution of NaOH. A pre-filtration is conducted, followed by a sterile filtration before the liquid being filled in glass vials and lyophilized. The lyophilization process starts with a freezing step, followed by a primary drying step under vacuum to remove the ice formed during freezing. In the last step of the lyophilization process, the secondary drying, any residual amounts of liquid which could not be removed by sublimation are removed under vacuum near or above ambient temperature to obtain a stable cake which can dissolve readily.

In another aspect of the invention, there is provided a process of preparing a formulation according to the present invention. The process comprises the step of adding or admixing an aqueous solution, e.g. a isotonic solution, to a compound and mannitol and optionally other pharmaceutically acceptable excipients such as e.g. an antioxidant, in a suitable vessel from a material which is non-reactive or substantially non-reactive with the formulation.

In one aspect of the present invention, the formulation is lyophilized. Lyophilization, or more commonly known as “freeze-drying”, is a process which extracts water from a solution to form a granular solid or powder. The process is carried out by freezing the solution and subsequently extracting any water or moisture by sublimation under vacuum. Lyophilization is particularly useful for developing pharmaceutical drug products that are reconstituted and administered to a patient by injection, for example drug products. In accordance with one embodiment of the present invention, the compound is lyophilized in the presence of a bulking agent and/or a non-volatile co-solvent to a pharmaceutically acceptable cake.

Methods for preparing the compositions and dosage forms are known or will be apparent to those skilled in the art. In other embodiments, a liquid or lyophilized dosage form of the present invention can be prepared according to the procedures set forth, for example, in Remington: The Science and Practice of Pharmacy, 20th Ed., Lippincott, Williams & Wilkins (2003); Pharmaceutical Dosage Forms, Volume 1: Tablets, 2nd Ed., Marcel Dekker, Inc., New York, N.Y. (1989); and similar publications.

In order to prepare a suitable pharmaceutically acceptable lyophilized cake of the compounds, an appropriate amount, e.g., conveniently a therapeutically effective amount of the compound is mixed with a water or an aqueous-based solvent, a nonvolatile cosolvent (optional) and a bulking agent to form a solution. The solution contains, e.g., a concentration of the bulking agent from about 0.1% to about 60% (w/v), e.g., 1% to about 50% or about 1% to 10% or about 3 to 5%, e.g. about 4%. In another embodiment, the formulation comprises about 40 mg/ml of the bulking agent. Suitable bulking agents have been described above. A preferred bulking agent in accordance with the present invention is mannitol. In one embodiment, the mannitol is D(-)mannitol.

Furthermore, the solution optionally contains, e.g., a concentration of the nonvolatile cosolvent from about 0.01% to about 75% (w/v), e.g., about 0.1% to about 50% or about 50% to about 75%, e.g., about 1% to about 25% or about 25% to about 75%. Optionally, a surfactant can also be added. The resulting solution is typically homogeneous and optically clear. In one group of embodiments, the solution does not comprise any solvents having a relatively high vapor pressure, e.g. lower alcohols, such as ethanol, isopropanol or tert-butanol. The concentration of the compound in the solution is preferably at least 3 mg/ml, preferably at least 4 mg/ml, more preferably at least 15 mg/ml. Typically, the concentration of the compound is between 4 mg/ml and 25 mg/ml, e.g. between 15 mg/ml and 23 mg/ml. In another preferred embodiment, the solution before lyophilization contains 4 mg/ml to 25 mg/ml such as, e.g., 10 mg/ml to 15 mg/ml or e.g., 15 mg/ml to 23 mg/ml of compound (free base equivalent) in 1% to 10% such as, e.g., 3% to 5% mannitol, or e.g. 4%. Though there may be additional excipients such as e.g. nonvolatile cosolvent or a surfactant present, in one preferred embodiment, the solution does not contain additional excipients.

Once mixed, the solution is filled into a container that is suitable for lyophilization, e.g., a glass vial. The lyophilization cycle typically includes the following steps: a freezing step, a primary drying step and a secondary drying step. In the freezing step, the solution is cooled. The temperature and duration of the freezing step is chosen such that all of the ingredients in the composition are completely frozen. For example, a suitable freezing temperature is approximately −40° C. The water in the formulation becomes crystalline ice. The balance of the formulation in the frozen state may be crystalline, amorphous or a combination thereof. In the primary drying step, the ice formed during freezing is removed by sublimation at sub-ambient temperatures under vacuum. For example, the chamber pressure used for sublimation can be from about 40 milliTorr to 400 milliTorr and the temperature be between −30° C. to −5° C. During the primary drying step, the formulation should be maintained in the solid state below the collapse temperature (“T_{c}”) of the formulation. The T_{c} is the temperature above which the freeze-dried-cake loses macroscopic structure and collapses during freeze-drying. For amorphous products the glass transition temperature (“T_{g}”) or for crystalline products the eutectic temperature (“Tₑ”) are approximately the same as T. In addition, the T_{g} for the maximally freeze concentrated solution (“T′_{g}”) is important to the development of lyophilization cycles because this represents the highest temperature that is safe for the composition for primary drying. After primary drying, any residual amounts of liquid which could not be removed by sublimation is removed by secondary drying, i.e., desorption. The temperature during secondary drying is near or greater than ambient temperature. After lyophilization, the pharmaceutical composition becomes a cake. Such a cake should be pharmaceutically acceptable. As used herein, a “pharmaceutically acceptable cake” refers to a non-collapsed solid drug product remaining after lyophilization that has certain desirable characteristics, e.g. pharmaceutically acceptable, long-term stability, a short reconstitution time, an elegant appearance and maintenance of the characteristics of the original solution upon reconstitution. The pharmaceutically acceptable cake can be solid, powder or granular material. The pharmaceutically acceptable cake may also contain up to five percent water by weight of the cake.

During the lyophilization process, the bulking agent will not sublime from the pharmaceutical composition. In the final pharmaceutically acceptable cake, the cake, e.g. comprises from about 0.1% to about 60% (w/w) of the bulking agent; e.g., from about 1% to about 50% (w/w); e.g., from about 1% to about 10% (w/w). Suitable bulking agents has been described above. Where the bulking agent is mannitol, the cake conveniently comprises of the mannitol from about 1% to about 10% (w/w), e.g., from about 3% to about 5% (w/w), e.g., from about 4% (w/w). The pharmaceutical composition or pharmaceutically acceptable cake will suitably contain between 0.1 mg and 250 mg of the therapeutic compound per unit dose, e.g., 0.1 mg, 1 mg, 5 mg, 10 mg, 15 mg, 17 mg, 20 mg, 23 mg, 25 mg, 50 mg, 100 mg, 120 mg, or 150 mg per unit dose. In some embodiments 10, 20, 30, 40, 50, 60, 70, 80, 90, 100, 110 or 120 mg per unit dose is used. As used herein, a “lyophilized pharmaceutical composition” refers to a non-collapsed solid drug product remaining after lyophilization that has certain desirable characteristics, e.g., pharmaceutically acceptable, long-term stability, a short reconstitution time, an elegant appearance and maintenance of the characteristics of the original solution upon reconstitution. The lyophilized pharmaceutical composition can be solid, powder or granular material. The lyophilized pharmaceutical composition may also contain up to five percent water by weight of the cake.

The lyophilized pharmaceutical composition cake can be reconstituted, e.g., for instant use. If all required components (e.g., buffer, isotonic agent) are present in the cake, sterile de-ionized water may be used for the reconstitution. Alternatively, an isotonic solution such as e.g. PLASMA-LYTE A® from Baxter or Ringer Acetate Solution from Baxter may be used for reconstitution. The reconstituted solution typically contains about 4 mg/ml to about 25 mg/ml such as, e.g., 15 mg/ml to 23 mg/ml of compound (free acid equivalent). Preferably, the reconstituted solution contains at least 3 mg/ml, at least 4 mg/ml or at least 15 mg/ml of compound. The reconstituted solution typically contains 1% to 50%, e.g., 1% to 10%, e.g., 3% to 5% of mannitol. The pH of the reconstituted solution is between 4 to 10, e.g., preferably 9 to 10, e.g. about 9.0 to about 9.5. In one embodiment, the pH is about 9.0 to about 9.1, 9.2, 9.3, 9.4, 9.5, 9.6, 9.7 or 9.8. In one embodiment, the pH is about 9.1 to about 9.2, 9.3, 9.4, 9.5, 9.6, 9.7 or 9.8. In one embodiment, the pH is about 9.2 to about 9.2, 9.3, 9.4, 9.5, 9.6, 9.7 or 9.8. In another group of embodiments, the pH of the liquid or lyophilized pharmaceutical formulation is about 9.0 to about 9.5. In one embodiment the pH of the liquid or lyophilized pharmaceutical formulation is about 9.1 to about 9.4. In one embodiment the pH of the liquid or lyophilized pharmaceutical formulation is about 9.3.

The dosage form to be administered will, in any event, contain a quantity of the therapeutic agent in a therapeutically effective amount for relief of the condition being treated when administered in accordance with the teachings of this invention. Typically, the compositions of the present invention comprise at least about 3 mg/mL by weight of the active agent (in whatever chosen form, measured as per its free acid form), and more typically from about 4 mg/mL to about 25 mg/mL. In some embodiments, about 10 mg/mL to about 15 mg/mL, or about 15 mg/mL to about 23 mg/mL of the active agent is used. One skilled in the art understands that the foregoing concentrations will vary depending upon the particular source of active agent utilized and the amount of active agent desired in the final formulation. In some embodiments, the formulation is not refrigerated and can be stored and used at room temperature. In other embodiments, the formulation can be refrigerated.

The dose and method of administration will vary from subject to subject and be dependent upon such factors as the type of mammal being treated, its sex, weight, diet, concurrent medication, overall clinical condition, the specific use for which the compound or pharmaceutical composition is employed, and other factors which those skilled in the medical arts will recognize.

### Methods of Administration

The compositions of the present invention are useful in therapeutic applications, e.g., for treating thrombosis. In addition, the compositions of the present invention offer advantages over compositions for injectable administration that do not contain an excipient described herein. In particular, because the excipient, co-solvent, pH adjusting agent or buffering agent or combination thereof in the compositions of the present invention can help increase the solubility of the active, the therapeutic agent can be administered with a higher dosage form and a larger amount of the therapeutic agent can be in systemic circulation in a substantially shorter period of time (e.g., reducing the time to onset of therapeutic activity) and at a substantially higher concentration than with compositions for injectable administration that do not contain the carrier.

The compositions of the present invention have particular utility in the area of human and veterinary therapeutics. The compositions of the present invention may be administered to deliver an active agent to any animal in need thereof, including, but not limited to, mammals, such as rodents, cows, pigs, dogs, cats, and primates, particularly humans. Generally, administered dosages will be effective to deliver picomolar to micromolar concentrations of the active agent to the appropriate site. Administration of the compositions of the present invention is preferably carried out via any of the accepted modes of injectable administration. In one group of embodiments, the liquid or lyophilized pharmaceutical formulation is administrable by intravenous (bolus and/or infusion), perivascular, subcutaneous, intramuscular, parenteral or intraperitoneal injection.

In a preferred embodiment, the formulation is suitable for intravenous administration. This form of administration is highly desirable in emergency situations. Furthermore, as no absorption process is involved, the dose or blood concentration of active agent may be obtained with greater accuracy and speed.

### Methods of Treatment

Methods for preventing or treating thrombosis in a mammal embraced by the invention administering a pharmaceutical composition of the invention as described above to a mammal, in particular, a human. Pharmaceutical compositions of the invention are suitable for use alone or as part of a multi-component treatment regimen for the prevention or treatment of cardiovascular diseases, particularly those related to thrombosis. For example, a compound or pharmaceutical composition of the invention may be used as a drug or therapeutic agent for any thrombosis, particularly a platelet-dependent thrombotic indication, including, but not limited to, acute coronary syndrome, ACS, acute myocardial infarction, unstable angina, chronic stable angina, transient ischemic attacks, strokes, peripheral vascular disease, preeclampsia/eclampsia, deep venous thrombosis, embolism, disseminated intravascular coagulation and thrombotic cytopenic purpura, thrombotic and restenotic complications following invasive procedures, e.g., angioplasty, carotid endarterectomy, post CABG (coronary artery bypass graft) surgery, vascular graft surgery, stent placements and insertion of endovascular devices and protheses, and hypercoagulable states related to genetic predisposition or cancers. In other groups of embodiments, the indication is selected from the group consisting of percutaneous coronary intervention (PCI) including angioplasty and/or stent, acute myocardial infarction (AMI), unstable angina (USA), coronary artery disease (CAD), transient ischemic attacks (TIA), stroke, peripheral vascular disease (PVD), Surgeries—coronary bypass, carotid endarectomy

Pharmaceutical compositions of the invention may also be used as part of a multi-component treatment regimen in combination with other therapeutic or diagnostic agents in the prevention or treatment of thrombosis in a mammal. In certain preferred embodiments, compounds or pharmaceutical compositions of the invention may be coadministered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, enoxaparin, glycoprotein (GP) 2b/3a inhibitors, aspirin, statins, angiotensin-converting enzyme (ACE) inhibitors or warfarin or anti-inflammatories (non-steriodal anti-inflammatories, cyclooxygenase II inhibitors). Co-administration may also allow for application of reduced doses of both the anti-platelet and the thrombolytic agents and therefore minimize potential hemorrhagic side-effects. Compounds and pharmaceutical compositions of the invention may also act in a synergistic fashion to prevent reocclusion following a successful thrombolytic therapy and/or reduce the time to reperfusion.

The compositions of the invention may be administered by injection in an effective amount within the dosage ranges described herein in a regimen of single or multiple (twice, etc.) daily or single or multiple weekly doses.

The pharmaceutical compositions of the invention may be utilized in vivo, ordinarily in mammals such as primates, (e.g., humans), sheep, horses, cattle, pigs, dogs, cats, rats and mice, or in vitro. The biological properties, as defined above, of a pharmaceutical composition of the invention can be readily characterized by methods that are well known in the art such as, for example, by in vivo studies to evaluate antithrombotic efficacy, and effects on hemostasis and hematological parameters.

The activity and characteristics of the compositions of the invention may be indicated in standard
a) human clinical trials, e.g. observing pharmacokinetic and pharmacodynamic parameters
b) animal trials e.g. observing pharmacokinetic and pharmacodynamic parameters

The compositions of the invention lead to an inter- and intra-patient reduced variability of Elinogrel, Elinogrel salt, for example potassium Elinogrel, or Elinogrel sodium.

The compositions of the invention are particularly useful for treatment or prevention of ADP-mediated disease or condition. As used herein, the term “ADP-mediated disease or condition” and the like refers to a disease or condition characterized by less than or greater than normal, ADP activity. An ADP-mediated disease or condition is one in which modulation of ADP results in some effect on the underlying condition or disease (e.g., a ADP inhibitor or antagonist results in some improvement in patient well-being in at least some patients).

In particular, the present compositions of the invention are useful for the treatment and prevention of acute coronary syndrome or in secondary prevention thereof.

The dose of the Elinogrel, Elinogrel salt, e.g. sodium Elinogrel salt, or Elinogrel crystalline or amorphous form, may vary depending on a variety of factors, for example the compound chosen, the particular condition to be treated and the desired effect. Dosages of Elinogrel salt or polymorph are to be calculated to correspond to the above mentioned dosages of Elinogrel free acid.

The terms “co-administration” or “combined administration” or the like as utilized herein are meant to encompass administration of the drug substance to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the drug substance and the active co-agent are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

The term “standard clinical study” comprises the conduct of such a study as regulated by health authorities e.g. Guidance for Industry, Bioavailability and Bioequivalence, Studies for Orally Administered Drug Products—General Considerations (Edition March 2003) from Division of Drug Information, HFD-240, Center for Drug Evaluation and Research, Food and Drug Administration, 5600 Fishers Lane, Rockville, Md. 20857

### Kits

Still another aspect of this invention is to provide a kit comprising a container in a single package, wherein the inventive pharmaceutical compositions thereof are used alone or in combination with pharmaceutically acceptable carriers to treat states, disorders, symptoms and diseases. Thus in one embodiment, the resultant formulation may be maintained under an inert atmosphere and is transferred to suitable containers, e.g. by a cannular system also under the inert atmosphere. Solvents other than water, when required, and other reagents may be chosen from medical grade reagents and solvents well known in the art. Formulations according to the invention may be packaged in containers. Containers may be chosen which are made of material. Glass containers may be used although it is preferred to use plastic containers, e.g. plastic infusion bags. In yet another embodiment, the invention provides an article of manufacture comprising a container enclosing a formulation disclosed herein. In one aspect, the article of manufacture is a pre-filled syringe. In yet another specific aspect, the pre-filled syringe is further contained within an injection device. In yet another specific aspect, the injection device is an auto-injector. In one embodiment of the present invention, there is provided a single dosage form suitable for e.g. intravenous administration comprising an effective amount of the compound and, optionally, further excipients commonly used in pharmaceutical compositions as e.g. described hereinabove. The single dosage form contains at least 3 mg/ml, preferably at least 4 mg/ml more preferably at least 15 mg/ml of the compound. In a single dose, conveniently, 10 mg, 15 mg, 17 mg, 20 mg, 23 mg, 25 mg, 40 mg, 50 mg, 75 mg, 100 mg, 120 mg, 125 mg, 150 mg, 175 mg, 200 mg or 250 mg are administered to a patent in need.

The following examples are intended for illustration only, are not intended to limit the scope of the invention. The contents of all U.S. patents and other references cited in this application are hereby incorporated by reference in the entirety.

### EXAMPLES

### Example 1

### Aqueous Solubilities of Elinogrel as Various Salt Forms and as a Function of pH

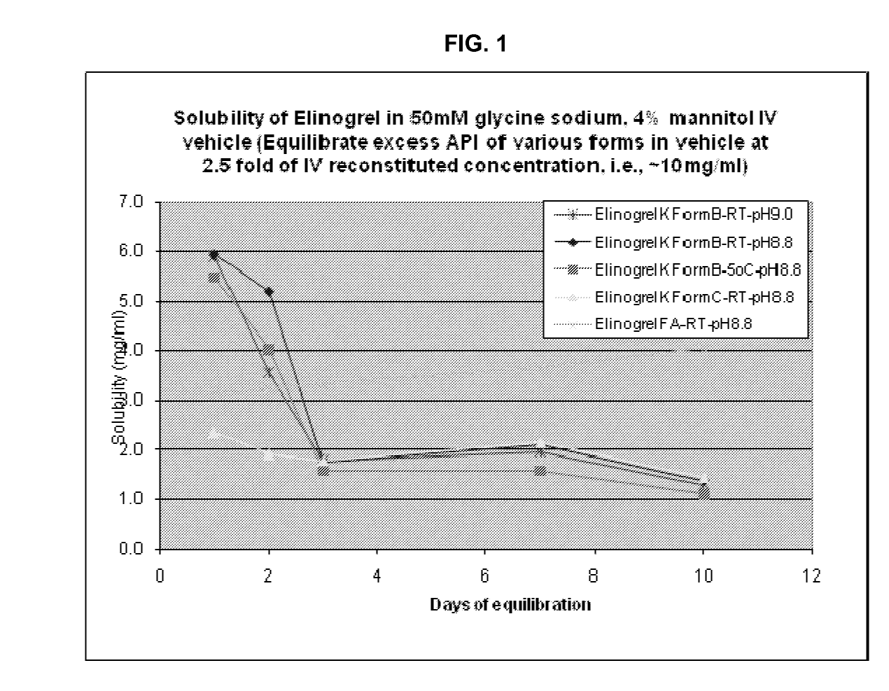

Three solid state forms of Elinogrel were tested: i.e., Form B (mono potassium hemi hydrate), Form C (hemi potassium trihydrate), and the Free Acid form. Excess Elinogrel (i.e., ˜10 mg/ml when fully dissolved) was equilibrated with the IV vehicle composed of 50 mM glycine sodium and 4% mannitol at RT or refrigerated. The pH was monitored and adjusted to the target range using NaOH or HCl. Results are shown in FIG. 1 and Table 1.

TABLE 1
Kinetic solubility of Forms B & C of Elinogrel potassium
in a liquid formulation containing mannitol and glycine buffer.

| | Solubility (mg/mL) | | | | |
|---|---|---|---|---|---|
| | Day 1 | Day 2 | Day 3 | Day 4 | Day 5 |
| Elinogrel K salt-Form B | 5.89 | 3.56 | 1.78 | 1.95 | 1.29 |
| RT-pH 9 | | | | | |
| Elinogrel K salt-Form B | 5.93 | 5.20 | 1.75 | 2.08 | 1.37 |
| RT-pH 8.8 | | | | | |
| Elinogrel K-Form B | 5.48 | 4.02 | 1.58 | 1.58 | 1.11 |
| 5° C. pH 8.8 | | | | | |
| Elinogrel K-Form C | 2.36 | 1.92 | 1.79 | 2.15 | 1.45 |
| RT-pH 8.8 | | | | | |
| Elinogrel free acid- | 3.14 | 3.23 | 3.33 | 2.65 | 4.05 |
| Form B-RT-pH9 | | | | | |

It was observed that while the kinetic solubility of elinogrel potassium salt is higher than that of the free acid, the thermodynamic solubility of the free acid form at pH 8.8-9.0 is higher. Since Elinogrel has a pKa of 3.5, at pH8.8-9.0, it presents itself essentially as Elinogrel sodium (dissociated).

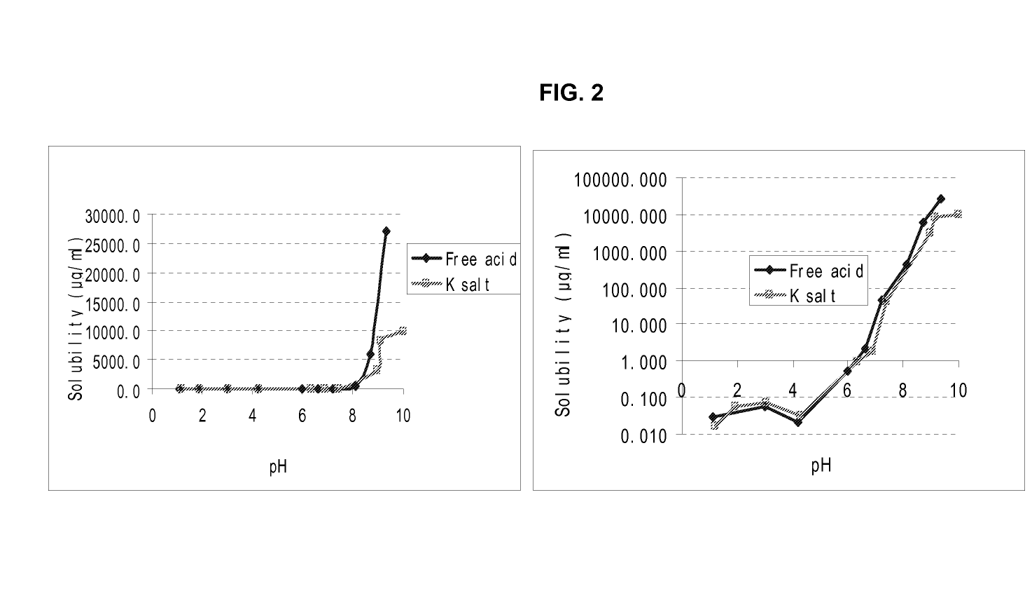

The thermodynamic solubility of Elinogrel potassium salt and free acid in pH range of 1-10 at 37° C. was also determined. Excess Elinogrel free acid or potassium salt (start from Form B) was equilibrated with its aqueous slurries at various pH under constant agitation. Different amounts of NaOH or HCl solution were used to adjust pH. Solubility was determined after 3 days by analyzing the elinogrel concentration in supernatant of the slurry after ultracentrifugation. Results were shown in FIG. 2 and Tables 2 and 3.

TABLE 2
The thermodynamic solubility of Elinogrel as a potassium salt at
different pH (adjusting pH with either NaOH or HCl) at 37° C..

| | Solubility |
|---|---|
| pH | (μg/mL) |
| 1.18 | 0.016 |
| 1.91 | 0.059 |
| 3.05 | 0.075 |
| 4.26 | 0.033 |
| 6.34 | 0.974 |
| 6.89 | 1.880 |
| 7.41 | 41.670 |
| 8.96 | 3′170.900 |
| 9.13 | 8′262.900 |
| 10.02 | 9′931.600 |

TABLE 3
The thermodynamic solubility of Elinogrel as a free acid at
different pH (adjusting pH with either NaOH or HCl) at 37° C.

| | Solubility |
|---|---|
| pH | (μg/mL) |
| 1.10 | 0.030 |
| 1.88 | 0.002 |
| 3.00 | 0.057 |
| 4.20 | 0.022 |
| 5.97 | 0.544 |
| 6.61 | 2.168 |
| 7.22 | 46.590 |
| 8.11 | 418.400 |
| 8.70 | 5′910.900 |
| 9.34 | 27′050.100 |

The consistent solubility values from free acid against potassium salt in pH range of 1-8 indicate that equilibrium was reached and the determined solubility is the thermodynamic solubility. The difference observed around pH 9-10 is due to different solubility of potassium salt and sodium salt of Elinogrel.

### Example 2

### Elinogrel Potassium Solubilities in Sodium Sulfobutyl Ether β-Cyclodextrin

A set of samples in 0, 10, 20, and 40% sodium sulfobutyl ether β-cyclodextrin with no pH adjustment were equilibrated for 12 days and concentrations of supernatants at 1, 3, 6, and 12 days were determined.

The solubility results of Elinogrel potassium at various sodium sulfobutyl ether β-cyclodextrin concentrations as a function of time (without pH adjustment) are shown in Table 4. The data show that the higher the sodium sulfobutyl ether β-cyclodextrin concentration, the higher the solubility. There is a continued decrease of solubility overtime, before it approaching plateau at each SBE-β-CD level.

TABLE 4
Elinogrel potassium concentration v · s time with no pH adjustment
in various sodium sulfobutyl ether β-cyclodextrin solutions (pH~7)

| | sodium sulfobutyl | Elinogrel |
|---|---|---|
| | ether β- | potassium conc. |
| Time (d) | cyclodextrin (%) | (mg/mL) |
| 1 | 40 | 50.59 |
| 3 | 40 | 51.47 |
| 6 | 40 | 34.19 |
| 12 | 40 | 19.52 |
| 1 | 20 | 28.12 |
| 3 | 20 | 28.47 |
| 6 | 20 | 21.55 |
| 12 | 20 | 11.50 |
| 1 | 10 | 16.3 |
| 3 | 10 | 16.46 |
| 6 | 10 | 5.63 |
| 12 | 10 | 5.89 |
| 1 | 0 | 6.97 |
| 3 | 0 | 7.77 |
| 6 | 0 | 2.56 |
| 12 | 0 | 2.74 |

Elinogrel potassium (Form C) solubility is 11.5 mg/mL in 20% sodium sulfobutyl ether β-cyclodextrin, which is approximately 4-fold as that in water. Therefore, sodium sulfobutyl ether β-cyclodextrin is able to increase Elinogrel potassium solubility by complexation.

The chemical stability of Elinogrel potassium in sodium sulfobutyl ether β-cyclodextrin solutions was tested and the results were summarized in Table 5. Elinogrel potassium chemical stability in Sodium sulfobutyl ether β-cyclodextrin solutions is slightly better than that in water.

TABLE 5
Elinogrel potassium chemical purity and related substances
in sodium sulfobutyl ether B-cyclodextrin solutions
at pH 7 and pH~10 (after 12 D at 25° C.)

| | Purity (%) | T = 12 d | T = 12 d |
|---|---|---|---|
| % CD (w/w) | T = 0 | at pH 7 | at pH 10 |
| 0 | 99.30 | 98.43 | 97.27 |
| 10 | 99.30 | 98.48 | 98.08 |
| 20 | 99.30 | 98.79 | 98.49 |
| 40 | 99.30 | 98.64 | 98.34 |

### Example 3

### Elinogrel Potassium Solubility Determinations in Co-Solvents

Excess Elinogrel potassium solids were added into ½ mL of 25% or 75% propylene glycol with 10% ethanol and the suspensions were equilibrated for 12 days at ambient temperature. Solubilities were determined by analyzing concentration of supernatants via HPLC.

Elinogrel potassium solubilities in propylene glycol (PG) and ethanol aqueous solutions are summarized in Table 6.

TABLE 6
Elinogrel potassium solubility in co-solvents
(no pH adjustment, 25° C., 12 d)

| Sample | Vehicle | S (mg/mL) | pH |
|---|---|---|---|
| Elinogrel potassium | 25% PG, 10% EtOH | 8 | 7.0 |
| (Form B) | | | |
| Elinogrel potassium | 75% PG, 10% EtOH | 21 | 7.3 |
| (Form B) | | | |

Higher solubility than that in water was achieved using propylene glycol and ethanol mixture as co-solvents.

### Example 4

### Elinogrel Sodium Hydrate Solubility Determinations

Elinogrel sodium hydrate was prepared by recrystallization from supersaturated elinogrel sodium solution in 5% sodium sulfobutyl ether β-cyclodextrin when stored at 4° C. overnight. The white to off-white wet solids were collected by centrifugation at 13,000 rpm for 5 min and supernatant removal. Based on DSC and polarized light microscopy, Elinogrel sodium hydrate lost water upon air-dry and converts to amorphous. To determine the Elinogrel sodium hydrate solubility, excess Elinogrel sodium crystalline hydrate was added into water or Elinogrel IV vehicle and concentration was determined at certain time points (1, 4, 8, 24 h, etc.) until it reached plateau. It was found that equilibrium was reached after 4 hours of agitation.

Elinogrel sodium hydrate solubilities in different vehicles are summarized in Table 7. Elinogrel sodium solubility is approximately 9-fold of that of Elinogrel potassium in water. Its solubility in sodium glycine buffer is slightly lower than that in water, likely due to a common-ion effect (Na).

TABLE 7
Elinogrel sodium hydrate solubility in various vehicles
(at ambient temperature if not indicated)

| Solvents | S (mg/mL) | pH |
|---|---|---|
| Water | 23 | 9.2 |
| Water (4° C.) | 13 | — |
| Na Glycine 50 mM, 4% mannitol | 16 | 9.3 |
| Na Glycine 10 mM, 4% mannitol | 17 | 9.3 |

### Example 5

### Elinogrel Sodium Stability in Solution

The stability of Elinogrel sodium solution at 15 mg/ml (as of Elinogrel free acid) was tested in 10 mM Na glycine, 4% mannitol, pH 9.3. Samples were stored at ambient temperature and 4° C. Aliquots were taken at day 0, 2, 4, 7 and 14 days and analyzed using stability indicating method. Appearance was also recorded at each time point.

Elinogrel sodium solution (15 mg/mL as of free acid) is chemically and physically stable in 10mM glycine, 4% mannitol, pH 9.3 at ambient temperature for 14 day (Table 8). Solution was clear at 4° C. for up to 7 day while precipitations were seen at 14 day due to solubility decrease at lower temperature.

TABLE 8
Elinogrel sodium Solution Stability (15 mg/mL)
in 10 mM glycine, 4% mannitol, pH 9.3

| | | Stored at Ambient Temp. | | | | Stored at 4° C. | | | |
|---|---|---|---|---|---|---|---|---|---|
| | T initial | 2 day | 4 day | 7 day | 14 day | 2 day | 4 day | 7 day | 14 day |
| Purity | 99.67 | 99.79 | 99.58 | 99.59 | 99.59 | 99.59 | 99.55 | 99.57 | 99.56 |
| (%) | | | | | | | | | |
| Assay | 100.1 | 100.6 | 100.1 | 101.4 | 102.4 | 101.3 | 101.5 | 94.9 | 96.3* |
| (%) | | | | | | | | | |

*Precipitates observed hence sample taken from supernatant for LC analysis.

### Example 6

### Dissolution Studies Using Elinogrel Potassium, Elinogrel Free Acid (to Form in-situ Sodium Salt) or Elinogrel Sodium as Starting Material

Dissolution studies conducted were summarized in Table 9 and results in Table 10.

TABLE 9
Conditions used in dissolution studies for elinogrel in various iv vehicles, pH, and scale.

| | | | | | | | Target | Solution |
|---|---|---|---|---|---|---|---|---|
| Starting | | Target | | | Agitation | Dissolution | conc. | stability |
| material | Vehicle | pH | Scale | | method | temperature | (mg/mL) | (Yes/No) |
| Elinogrel | 50 mM | 9.0 | 1 | L | internal | RT and | 4 | Y (4° C., |
| potassium | glycine, | | | | impeller | 50° C. | | 3 mo) |
| | 4% mannitol | | | | | | | |
| Elinogrel | 50 mM | 9.0, 9.3 | 250 | mL | internal | 50° C. | 20 | N |
| free acid | glycine, | and 9.5 | | | impeller | | | |
| | 4% mannitol | | | | | | | |
| Elinogrel | 50 mM | 9.5, 9.7 | 1 | mL | rocker or | RT and | 15 | Y (ambient & |
| free acid | glycine, | and | | | frequent | 50° C. | | 4° C., 15 d) |
| | 4% mannitol | 10.0 | | | vortex | | | |
| Elinogrel | 10 mM | 9.3 | 50 mL, 2 L | | gentle | RT | 15 | Y (ambient & |
| sodium | glycine, | | or 140 L | | swirl | | | 4° C., 2 wk) |
| | 4% mannitol | | | | w/vortex | | | |
| | | | | | or impeller | | | |
| Elinogrel | 10 mM | 9.3 | 100 | mL | Magnet | RT | 15 | y |
| free acid | glycine, | | | | stirrer* | | | |
| | 4% mannitol | | | | | | | |

*In this case, first sodium hydroxide was dissolved in ca 90 ml of water to obtain a pH of ca. 11.2. After the addition of Elinogrel at room temperature, the pH dropped in ca. 55 minutes to pH 10. Upon addition of mannitol and glycine the final 15 mg/ml Elinogrel solution was obtained containing 10 mM of glycine, 4% of mannitol having a pH of 9.2.

Elinogrel potassium (Form B) dissolution was tested at either ambient temperature or 50° C. at 1 L scale. Dissolution time was recorded. After dissolution completion, samples were taken before and after filtration through a 0.22 μm filter. All samples were stored at 4° C. to observe if precipitates formed over time.

Dissolution time for forming in-situ sodium salt using Elinogrel free acid as a starting material was performed in 50 mM Glycine and 4% mannitol (pH 9.0, 9.3 and 9.5) at 50° C. to have a final concentration of 20 mg/mL as of free acid and final volume 250 mL. Exact amount of NaOH in 1N solution was added for Elinogrel free acid conversion to Elinogrel sodium. If solids did not completely dissolve at 50° C. in 6 hours, then excess amount of NaOH was added to increase pH to expedite dissolution. Around 1 mL scale experiments at pH range 9.5 to 10.0 (target concentration 15 mg/mL) were set up and dissolution time at ambient and 50° C. was recorded.

Elinogrel sodium dissolution was performed in 10 mM glycine, 4% mannitol to have a final concentration of 15 mg/mL and pH 9.3.

Dissolution time was summarized in Table 10.

TABLE 10
Dissolution time: Elinogrel potassium and Elinogrel free acid in 50 mM
glycine, 4% mannitol; Elinogrel sodium in 10 mM glycine, 4% mannitol

| | | | | | Conc. by |
|---|---|---|---|---|---|
| Starting | Dissolution temp, | Dissolu- | | Final | HPLC |
| material | mixing mtd & scale | tion time | | pH | (mg/mL) |
| Elinogrel | RT, impeller, 1 L | 3.5 | h | 9.3 | 3.9 |
| potassium | | | | | |
| Elinogrel | 50° C., impeller, | 35 | min | 9.1 | 3.9 |
| potassium | 1 L | | | | |
| Elinogrel | 50° C., impeller, | not | | — | — |
| free acid | 250 mL | dissolved | | (targeted 9.0) | (targeted 20) |
| Elinogrel | 50° C., impeller, | not | | — | — |
| free acid | 250 mL | dissolved | | (targeted 9.3) | (targeted 20) |
| Elinogrel | 50° C., impeller, | 3 | h | 9.5 | — |
| free acid | 250 mL | | | | (targeted 20) |
| Elinogrel | RT, rocker, 1 mL | >4 | h | 9.5 | 15.1 |
| free acid | | | | | |
| Elinogrel | RT, rocker, 1 mL | 2 | h | 9.7 | 15.4 |
| free acid | | | | | |
| Elinogrel | RT, rocker, 1 mL | 45 | min | 10.0 | 15.3 |
| free acid | | | | | |
| Elinogrel | 50° C., vortex, 1 | 55 | min | 9.5 | 15.2 |
| free acid | mL | | | | |
| Elinogrel | 50° C., vortex, 1 | 22 | min | 9.8 | 15.3 |
| free acid | mL | | | | |
| Elinogrel | 50° C., vortex, 1 | 10 | min | 10.0 | 15.2 |
| free acid | mL | | | | |
| Elinogrel | RT, stirring, 100 | 55 | min | 9.2 | 15.0 |
| free acid | mL | | | | |
| Elinogrel | Ambient, swirl/ | <2 | min | 9.3 | 15.6 |

| | | | | |
|---|---|---|---|---|
| sodium | vortex, 50 mL | | | |
| Elinogrel | Ambient, impeller, | <15 | 9.3 | 15.0 |
| sodium | 2 L | minutes | | |
| Elinogrel | Ambient, impeller, | <60 | 9.3 | 15.0 |
| sodium | 140 L at pH 9.0 | minutes | | |

Dissolution time of Elinogrel potassium in 50 mM glycine, 4% mannitol was determined at both ambient temperature and 50° C. at 1 L scale. It was observed that significant longer dissolution time was required at ambient temperature, i.e., 3.5 hr vs. 35 min at 50° C. Therefore, heating is needed during Elinogrel potassium IV solution compounding to facilitate dissolution.

As Elinogrel sodium salt may be made in-situ by starting with Elinogrel free acid and then adjusting pH higher using NaOH, dissolution experiments using Elinogrel free acid as starting material were set up at the pH range from 9.0 to 9.5 at 250 mL scale. The results show that even with consistent heating at 50° C., Elinogrel free acid dissolves at pH 9.5 in about 3 hours. At both pH 9.0 and 9.3, Elinogrel free acid did not fully dissolve overnight with agitation.

Elinogrel sodium could also be formed in situ from elinogrel free acid at higher pHs. Suspension at pH 9.0 was added with extra NaOH to increase the pH to 10.3 and solids were dissolved in 1 hour. 1N HCl was then used to adjust pH back to 9.0. The resulted solution was stored at 25° C. overnight and needle/blade-shaped crystals precipitated overnight. It is considered that the precipitates are Elinogrel sodium hydrate and its concentration was reduced to 6 mg/mL. This is possibly due to the common-ion (Na) effect (around 40 mM sodium chloride generated during pH adjustment on top of 50 mM sodium glycine).

A stable Elinogrel sodium solution could be formed in situ from elinogrel free acid at higher pHs. A suspension was added with NaOH to increase the pH to 11.2 and solids were dissolved in 55 minutes, while the pH dropped to 10. Upon addition of mannitol and glycine the final 15 mg/ml Elinogrel solution was obtained containing 10 mM of glycine, 4% of mannitol having a pH of 9.2. The resulted solution was stored at room temperature for more than 3 days without precipitation. It was demonstrated that the purity of Elinogrel was not impacted by the pH excursion.

Dissolution studies of Elinogrel free acid to form sodium salt in situ at pH 9.5 to 10 were performed at 1 mL scale, 15 mg/mL. Results show that the higher the pH, the shorter the dissolution time. In addition, heat is still needed for this higher pH range 9.5-10 and elevated temperature improved Elinogrel free acid dissolution significantly (Table 7). No precipitations were seen for all samples stored at 4° C. or ambient temperature for fifteen days.

Elinogrel sodium dissolution was also performed to have a target concentration of 15 mg/mL (as free acid) and pH 9.3 in 10 mM glycine and 4% mannitol. Elinogrel sodium was dissolved completely in less than 2 min for 50 mL scale with gentle swirl/vortex or less than 60 minutes for up to 140 L scale with impeller agitation at ambient temperature. This approach offers significant advantage due to ease of processing.

### Example 7

### Elinogrel IV Lyophilized Formulation & Process

One IV drug formulation comprises a lyophilized powder for reconstitution, 20 mg of Elinogrel per vial. The product is reconstituted with 5 mL of water for injection (WFI) within 24 hours prior to dose administration. Each vial (before lyophilization) contains the following:

| Component | ~mg/vial |
|---|---|
| Elinogrel potassium | 20.40 |
| (as of free acid) | |
| Glycine* | 19.13 |
| D(-)Mannitol | 204.0 |
| Water for injection | Q.S. |

*The pH was adjusted to 9.0 ± 0.2 using NaOH in bulk solution prior to lyophilization. The glycine sodium is 50 mM in bulk solution (prior lyophilization) or post reconstitution.

The process for making the solution (prior filling and lyophilization) is outlined below. About 88% WFI is weighed and added to a stainless steel or glass pot, and heated to ˜45° C. The temperature is maintained below 45° C. Glycine is added and mixed to dissolve. Mannitol is added and mixed to dissolve. pH is adjusted to 9.0±0.2 with 1N NaOH or HCl. Compound is added and mixed to dissolve while heating the bulk solution (40-45° C.). The pH is confirmed and adjusted to 9.0±0.2 if necessary. Heating is discontinued. QS is targeted with WFI. The final theoretical concentration of Elinogrel in bulk solution is 4mg/ml (as of free acid equivalent). A bioburden sample is obtained. Pre-filtration is conducted (0.45 μm followed by 0.22 μm), and the bulk solution is held overnight if required by manufacturing schedule. The solution is double sterile filtered (double 0.22 μm) before being lyophilized. The lyophilization process is composed of commonly adopted procedure that involves freezing, primary drying, and secondary drying.

### Example 8

### Elinogrel IV Lyophilized Formulation & Process

One IV drug formulation comprises a lyophilized powder for reconstitution, 80mg of Elinogrel per vial. The product is reconstituted with 8 mL of WFI within 24 hours prior to dose administration. Each vial (before lyophilization) contains the following:

| Component | mg/mL | mg/vial¹ |
|---|---|---|
| Elinogrel sodium | 10.42² | 83.36³ |
| Glycine* | 0.75 | 6.0 |
| D(−)Mannitol | 40 | 320 |
| Water for injection | Q.S. | Q.S. |

*The pH was adjusted to 9.3 using NaOH in bulk solution prior to lyophilization. The glycine sodium is 10 mM in bulk solution (prior lyophilization) and post reconstitution.
¹This composition refers to the nominal volume of 8.0 mL, the overfill of 0.4 ml is not considered.
²10.42 mg PRT128. Na corresponds to 10 mg of the free acid
³83.36 mg PRT128. Na corresponds to 80 mg of the free acid

The process for making the solution (prior filling and lyophilization) is outlined below. About 85% WFI is weighed and added to a stainless steel vessel at ambient temperature. Glycine is added and mixed to dissolve. Mannitol is added and mixed to dissolve. pH is adjusted to 9.0±0.1 with 1N NaOH. Compound is added and mixed to dissolve. pH is adjusted to 9.3±0.1. The final theoretical concentration of Elinogrel in bulk solution is 15 mg/ml (as of free acid equivalent). Pre-filtration is conducted using a 0.2 μm rated Supor EAV polyethersulfone filter, followed by a double sterile filtration (0.22 μm twice) before being filled in 20R glass vials and lyophilized. The lyophilization process is composed of a procedure that involves freezing, primary drying, and secondary drying.

After equilibration the vials at 5° C., the shelves are cooled down to -19° C. at a rate of 0.6° C./min and held for 3 hours at −19° C. Then the shelves are cooled down to −40° C. at a speed of 1° C./min and kept at −40° C. for 3 hours. After two hours at −40° C., the primary drying is started by reducing the pressure to 0.5 mbar and after three hours at −40° C., the temperature is raised to 0° C. at a speed of 1° C./min and kept for 30 H at 0° C. for the rest of the primary drying. For the secondary drying the pressure is reduced to 0.1 mbar and the temperature is slowly raised to 40° C. (0.5° C./min) and kept for 10 hours at 40° C. After the secondary drying the lyophilization cycle is ended by bringing the shelf temperature down to 20° C. and the pressure up to to 850 mbar followed by the closing the vials.

### Example 9

### Elinogrel IV Lyophilized Formulation & Process

One IV drug formulation comprises a lyophilized powder for reconstitution, 120 mg of Elinogrel per vial. The product is reconstituted with 8 mL of WFI within 24 hours prior to dose administration. Each vial (before lyophilization) contains the following:

| Component | mg/mL | mg/vial¹ |
|---|---|---|
| Elinogrel sodium | 15.63² | 125.04³ |
| Glycine* | 0.75 | 6.0 |
| D(−)Mannitol | 40 | 320 |
| Water for injection | Q.S. | Q.S. |

*The pH was adjusted to 9.3 using NaOH in bulk solution prior to lyophilization. The glycine sodium is 10 mM in bulk solution prior lyophilization and post reconstitution.
¹This composition refers to the nominal volume of 8.0 mL, the overfill of 0.4 ml is not considered.
²15.63 mg Elinogrel sodium corresponds to 15 mg of the free acid
³125.04 mg Elinogrel sodium corresponds to 120 mg of the free acid

### Example 10

### Rabbit Vein and Tissue Irritation Study with Elinogrel at 4.0 and 16.7 mg/mL

Male New Zealand White rabbits were utilized in this study. Rabbits received 3 injections:

Intravenous (IV): 1.0 mL in left marginal ear vein;

Perivascular: 0.5 mL near the right marginal ear vein; and

Subcutaneous (SC): 1.0 mL in the scapular region of the dorsal back.

There were four rabbits per each of the following dose groups.

Normal Saline: 0.9% sodium chloride

Vehicle: 10 mM sodium glycine, 4% mannitol, pH 9.31

Elinogrel (Na salt) at 16.7 mg/mL in 10 mM glycine, 4% mannitol, pH 9.33

Elinogrel (K salt) at 4.0 mg/mL in 50 mM glycine, 4% mannitol, pH 9.38

Injection sites were observed at 1, 3, and 6 hours post-injection and then twice a day for 5 days. On day 5, tissues (ears and skin) were harvested and processed for histological examination of the injection site. The intravenous, perivascular and subcutaneous administration of Elinogrel at 4.0 or 16.7 mg/mL yielded similar animal observation as the vehicle and saline control and the formulations were well tolerated.

### Example 11

### Clinical Testing of Elinogrel Potassium Formulation

The elinogrel potassium IV formulation has been tested in multiple clinical studies (06-106, 07-112, and 07-113), total >100 subjects. The tested doses are in the range of 10 mg to 60 mg per subject. The doses were administered either as 20 min i.v. infusion or i.v. bolus. All tested doses have been well tolerated. There have been no deaths, 2 SAEs (vs. 8 SAEs in the placebo comparator arm), and 1 discontinuation due to a mild adverse event associated with iv elinogrel administration.

### Example 12

### Clinical Testing of elinogrel Sodium Salt Formulation

The elinogrel sodium IV formulation according to Example 9 has been tested in clinical studies in humans. The tested dose was 60 mg per subject, administered as an i.v. bolus dose. The intravenous administration of 60 mg Elinogrel gave a rapid onset of P2Y12 inhibition measured using VerifyNow cartridges with obtained peak P2Y12 inhibition within one hour after administration.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A liquid pharmaceutical composition comprising at least about 3 mg/ml Elinogrel, or a pharmaceutically acceptable salt thereof.

2. A lyophilized pharmaceutical composition comprising at least about 3 mg/ml Elinogrel, or a pharmaceutically acceptable salt thereof after reconstitution in solution.

3.-34. (canceled)

35. A liquid dosage form comprising at least about 3 mg/ml Elinogrel or a pharmaceutically acceptable salt thereof.

36. A lyophilized dosage form comprising at least about 3 mg/ml Elinogrel or a pharmaceutically acceptable salt thereof after reconstitution.

37. A liquid dosage form comprising up to about 15 mg/ml Elinogrel or a pharmaceutically acceptable salt thereof.

38. A lyophilized dosage form comprising up to about 15 mg/ml Elinogrel or a pharmaceutically acceptable salt thereof after reconstitution.

39.-47. (canceled)

48. A method of treating or preventing a thrombotic condition in a mammal in need thereof, which method comprises intravenous administering to said mammal an effective amount of a liquid pharmaceutical composition comprising at least about 3 mg/ml Elinogrel.

49. A method of producing a liquid pharmaceutical composition comprising at least about 3 mg/ml Elinogrel, or a pharmaceutically acceptable salt thereof; by contacting a) Elinogrel or a pharmaceutically acceptable salt thereof in solution, with b) at least one pharmaceutically acceptable excipient.

50. A method of producing a lyophilized pharmaceutical composition comprising at least about 3 mg/ml Elinogrel, or a pharmaceutically acceptable salt thereof after reconsitution; comprising a) forming a solution comprising at least about 3 mg/ml Elinogrel in an aqueous solvent, optionally a co-solvent and a bulking agent and (b) lyophilizing said solution to form a lyophilized pharmaceutical composition.

51. A method of treating or preventing a thrombotic condition in a mammal in need thereof, which method comprises intravenous administering to said mammal an effective amount of a liquid pharmaceutical composition comprising up to about 15 mg/ml Elinogrel.

52. A method of producing a liquid pharmaceutical composition comprising up to about 15 mg/ml Elinogrel, or a pharmaceutically acceptable salt thereof; by contacting a) Elinogrel or a pharmaceutically acceptable salt thereof in solution, with b) at least one pharmaceutically acceptable excipient.

53. A method of producing a lyophilized pharmaceutical composition comprising up to about 15 mg/ml Elinogrel, or a pharmaceutically acceptable salt thereof after reconsitution; comprising a) forming a solution comprising up to about 15 mg/ml Elinogrel in an aqueous solvent, optionally a co-solvent and a bulking agent and (b) lyophilizing said solution to form a lyophilized pharmaceutical composition.

54. A kit of comprising a dosage form according to any one of claims 35 to 37 in a suitable container.

55. (canceled)
